(19) United States
(12) Patent Application Publication
(10) Pub. No.: US 2021/0361917 A1
(43) Pub. Date: Nov. 25, 2021

(54) External Catheter Assembly

(71) Applicants: Linda Edwards, Queens, NY (US); Charina Perez, Queens, NY (US)

(72) Inventors: Linda Edwards, Queens, NY (US); Charina Perez, Queens, NY (US)

(21) Appl. No.: 16/880,651

(22) Filed: May 21, 2020

(51) Int. Cl. A61M 25/09; A61F 5/453; A61F 5/44; A61F 5/455

(52) U.S. Cl. CPC A61M 25/09041; A61F 5/453; A61F 5/4408; A61M 2025/0177; A61M 2210/1092; A61M 2210/1096; A61M 2210/1085; A61F 5/455

(57) ABSTRACT

An external catheter assembly for facilitating non-invasive catheterization includes a male catheter that is wrappable around a penis to capture urine from the penis. A first tube is fluidly coupled to the male catheter to receive the urine from the penis. A first collection bag is fluidly coupled to the first tube to capture the urine from the penis. A female catheter is positionable within the labia minora of a female patient to capture urine from the female patient. A second tube is fluidly coupled to the female catheter to receive the urine from the female patient. A second collection bag is fluidly coupled to the second tube to capture the urine from the female patient.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to catheter devices and more particularly pertains to a new catheter device for facilitating non-invasive catheterization.

### (2) Description of Related Art Including Information Disclosed Under 37 CFR 1.97 and 1.98

The prior art relates to catheter devices. The prior art discloses an adhesive, external catheter for use on a female. Additionally, the prior art discloses a variety of external collectors, including a cup and a sleeve, that can be positioned against a labia or around a penis for external catheterization. The prior art discloses a non-invasive catheter that is integrated into a garment for wearing. In each instance the prior art discloses either a male catheter or a female catheter, but not both.

### BRIEF SUMMARY OF THE INVENTION

An embodiment of the disclosure meets the needs presented above by generally comprising a male catheter that is wrappable around a penis to capture urine from the penis. A first tube is fluidly coupled to the male catheter to receive the urine from the penis. A first collection bag is fluidly coupled to the first tube to capture the urine from the penis. A female catheter is positionable within the labia minora of a female patient to capture urine from the female patient. A second tube is fluidly coupled to the female catheter to receive the urine from the female patient. A second collection bag is fluidly coupled to the second tube to capture the urine from the female patient.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an external catheter assembly according to an embodiment of the disclosure.

FIG. 2 is a front view of a male catheter of an embodiment of the disclosure.

FIG. 3 is a perspective in-use view of a male catheter of an embodiment of the disclosure.

FIG. 4 is a front view of a female catheter of an embodiment of the disclosure.

FIG. 5 is a perspective in-use view of a female catheter of an embodiment of the disclosure.

### DETAILED DESCRIPTION OF THE INVENTION

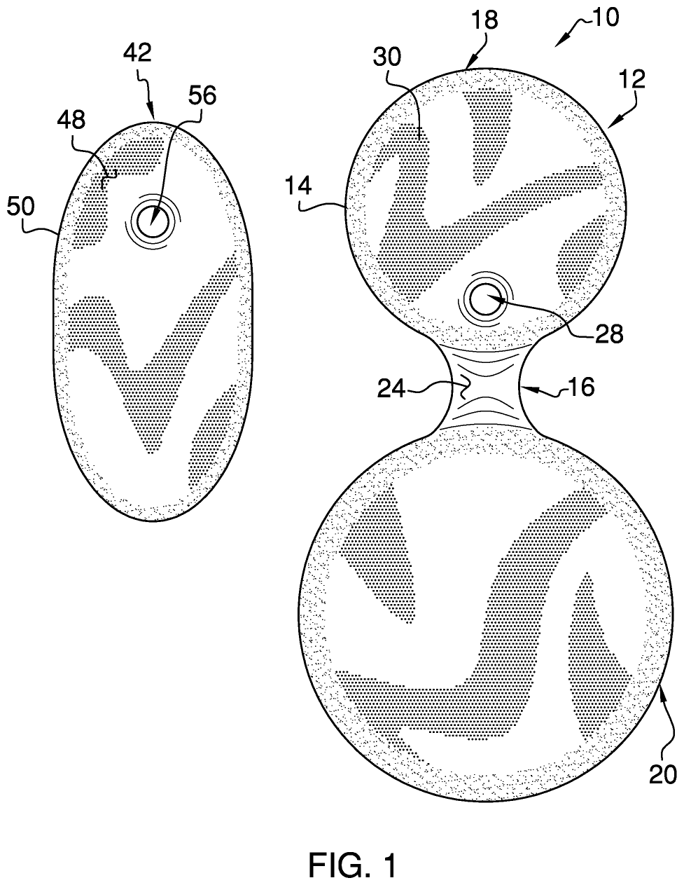

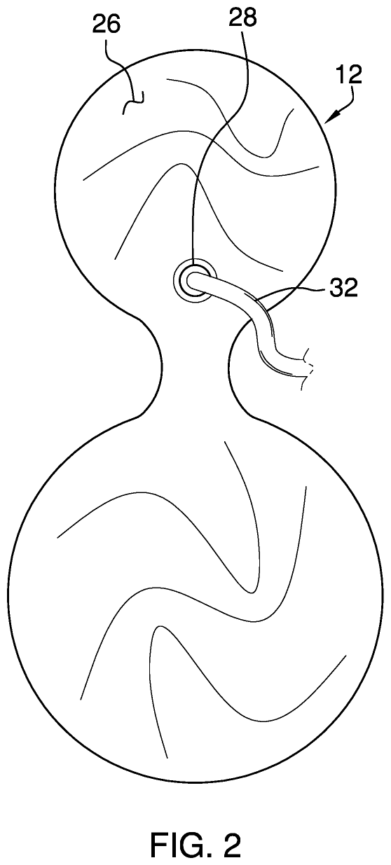

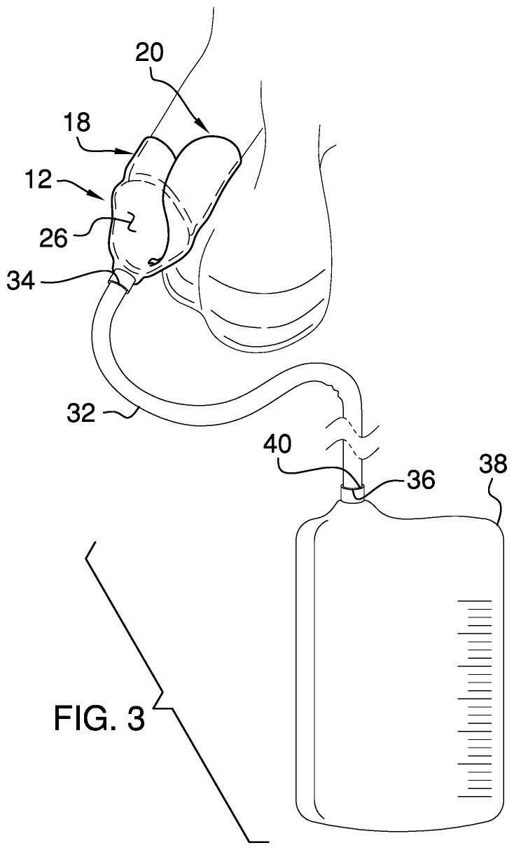

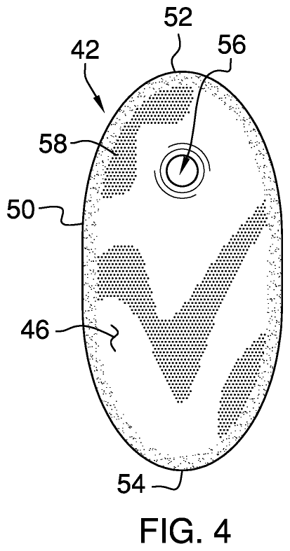

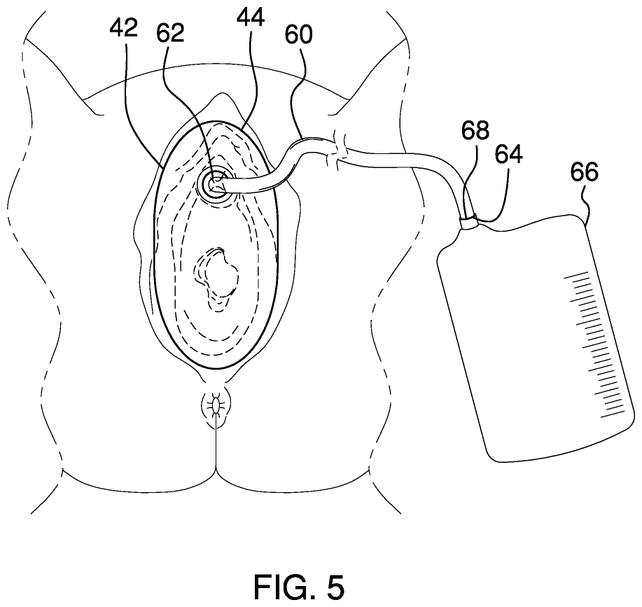

With reference now to the drawings, and in particular to FIGS. 1 through 5 thereof, a new catheter device embodying the principles and concepts of an embodiment of the disclosure and generally designated by the reference numeral 10 will be described.

As best illustrated in FIGS. 1 through 5, the external catheter assembly 10 generally comprises a male catheter 12 that has an undulating perimeter edge 14 which defines a peninsula 16 extending between a first lobe 18 and a second lobe 20. The male catheter 12 is wrappable around a tip of a penis 22 having the first lobe 18 positioned on top of the penis 22 and having the second lobe 20 positioned on the bottom of the penis 22. In this way wherein the male catheter 12 can capture urine from the penis 22. The male catheter 12 has a first surface 24 and a second surface 26 having the perimeter edge 14 extending therebetween.

The first lobe 18 has an aperture 28 extending through the first surface 24 and the second surface 26. In this way the aperture 28 is aligned the external urethral orifice in the penis 22 thereby facilitating urine to pass through the aperture 28. The aperture 28 is positioned adjacent to an intersection between the first lobe 18 and the peninsula 16. Additionally, the male catheter 12 is infused with aloe vera for reducing irritation caused by wearing the male catheter 12.

A first adhesive layer 30 is bonded to the male catheter 12 and the first adhesive layer 30 adhesively engages the penis 22 when the male catheter 12 is wrapped around the penis 22 to retain the male catheter 12 on the penis 22. The first adhesive layer 30 is positioned on the first surface 24 of the male catheter 12 and the first adhesive layer 30 completely covers the first surface 24. The first adhesive layer 30 may comprise an acrylic adhesive or other surgical grade adhesive.

A first tube 32 is fluidly coupled to the male catheter 12 to receive the urine from the penis 22. The first tube 32 has a first end 34 and a second end 36, and the aperture 28 in the male catheter 12 insertably receives the first end 34 having the first tube 32 being bonded to the male catheter 12. A first collection bag 38 is fluidly coupled to the first tube 32 to capture the urine from the penis 22. The first collection bag 38 has an inlet 40 integrated therein and the inlet 40 has the second end 36 of the first tube 32 being fluidly coupled thereto.

A female catheter 42 is positionable within the labia minora 44 of a female patient to capture urine from the female patient. The female catheter 42 has a primary surface 46, a secondary surface 48 and an outer edge 50 extending therebetween, and the outer edge 50 is continuously arcuate about a center point of the female catheter 42. The female catheter 42 is longitudinally elongated such that the female catheter 42 has an ovoid shape having a first end 52 and a second end 54. In this way the female catheter 42 can cover the vaginal opening and the urethra when the female catheter 42 is worn.

The female catheter 42 has an aperture 56 extending through the primary surface 46 and the secondary surface 48 to have urine pass therethrough. The aperture 56 in the female catheter 42 is aligned with a longitudinal axis of the female catheter 42. Additionally, the aperture 56 in the female catheter 42 is positioned closer to the first end 52 than the second end 54 of the ovoid shape defined by the female catheter 42. The female catheter 42 is infused with aloe vera for reducing irritation caused by wearing the female catheter 42.

A second adhesive layer 58 is provided and the second adhesive layer 58 is bonded to the female catheter 42. The second adhesive layer 58 adhesively engages the labia minora 44 when the female catheter 42 is positioned thereagainst to retain the female catheter 42 on the labia minora 44. The second adhesive layer 58 is positioned on the secondary surface 48 of the female catheter 42 and the second adhesive layer 58 completely covers the secondary surface 48. The second adhesive layer 58 may comprise an acrylic adhesive or other surgical grade adhesive.

A second tube 60 is fluidly coupled to the female catheter 42 to receive the urine from the female patient. The second tube 60 has a first end 62 and a second end 64, and the aperture 56 in the female catheter 42 insertably receives the second end 64 of the second tube 60 having the second tube 60 being bonded to the female catheter 42. A second collection bag 66 is fluidly coupled to the second tube 60 to capture the urine from the female patient. The second collection bag 66 has an inlet 68 integrated therein, and the second end 64 of the second tube 60 is fluidly coupled to the inlet 68 in the second collection bag 66.

In use, the male catheter 12 is wrapped around the penis 22 such that the aperture 28 in the male catheter 12 is aligned with the external urethral opening in the penis 22. In this way the urine passes through the first tube 32 for collection in the first collection bag 38. Thus, a male patient can be catheterized in a non-invasive procedure, compared to a foley catheter for example, thereby reducing the risk of infections related to invasive catheterization. The female catheter 42 is positioned on the labia minora 44 thereby facilitating the aperture 56 in the female catheter 42 to be aligned with the female patient's urethra. In this way urine passes through the second tube 60 for collection in the second collection bag 66. The female catheter 42 includes the same benefits as the male catheter 12 with respect to being non-invasive.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An external catheter assembly being configured to be externally applied to a patient for urine collection, said assembly comprising:
a male catheter having an undulating perimeter edge that defines a peninsula extending between a first lobe and a second lobe, said male catheter being wrappable around a penis having said top lobe being positioned on top of the penis and having said bottom lobe being positioned on the bottom of the penis wherein said male catheter is configured to capture urine from the penis;
a first adhesive layer being bonded to said male catheter, said first adhesive layer adhesively engaging the penis when said male catheter is wrapped around the penis to retain said male catheter on the penis;
a first tube being fluidly coupled to said male catheter wherein said first hose is configured to receive the urine from the penis;
a first collection bag being fluidly coupled to said first tube wherein said first collection bag is configured to capture the urine from the penis;
a female catheter being positionable within the labia minora of a female patient wherein said female catheter is configured to capture urine from the female patient;
a second adhesive layer being bonded to said female catheter, said second adhesive layer adhesively engaging the labia minora when said female catheter is positioned thereagainst to retain said female catheter on the labia minora;
a second tube being fluidly coupled to said female catheter wherein said second hose is configured to receive the urine from the female patient; and
a second collection bag being fluidly coupled to said second tube wherein said second collection bag is configured to capture the urine from the female patient.

2. The assembly according to claim 1, wherein said male catheter has a first surface and a second surface having said perimeter edge extending therebetween, said first lobe having an aperture extending through said first surface and said second surface wherein said aperture is configured to be aligned the external urethral orifice in the penis thereby facilitating urine to pass through said aperture, said aperture being positioned adjacent to an intersection between said first lobe and said peninsula, said male catheter being infused with aloe vera for reducing irritation caused by wearing said male catheter.

3. The assembly according to claim 2, wherein said first adhesive layer is positioned on said first surface of said male catheter, said first adhesive layer completely covering said first surface.

4. The assembly according to claim 3, wherein said first tube has a first end and a second end, said aperture in said male catheter insertably receiving said first end having said first tube being bonded to said male catheter.

5. The assembly according to claim 4, wherein said first collection bag has an inlet being integrated therein, said inlet having said second end of said tube being fluidly coupled thereto.

6. The assembly according to claim 1, wherein said female catheter has a primary surface, a secondary surface and an outer edge extending therebetween, said outer edge being continuously arcuate about a center point of said female catheter, said female catheter being longitudinally elongated such that said female catheter has an ovoid shape having a first end and a second end wherein said female catheter is configured to cover the vaginal opening and the urethra when said female catheter is worn.

7. The assembly according to claim 6, wherein said female catheter has an aperture extending through said primary surface and said secondary surface wherein said aperture in said female catheter is configured to have urine pass therethrough, said aperture in said female catheter being aligned with a longitudinal axis of said female catheter, said aperture in said female catheter being positioned closer to said first end than said second end of said ovoid shape defined by said female catheter, said female catheter being infused with aloe vera for reducing irritation caused by wearing said female catheter.

8. The assembly according to claim 7, wherein said second adhesive layer is positioned on said secondary surface of said female catheter, said second adhesive layer completely covering said secondary surface.

9. The assembly according to claim 8, wherein said second tube has a first end and a second end, said aperture in said female catheter insertably receiving said second end of said second tube having said second tube being bonded to said female catheter.

10. The assembly according to claim 9, wherein said second collection bag has an inlet being integrated therein, said inlet on said second collection bag having said second end of said second tube being fluidly coupled thereto.

11. An external catheter assembly being configured to be externally applied to a patient for urine collection, said assembly comprising:
a male catheter having an undulating perimeter edge that defines a peninsula extending between a first lobe and a second lobe, said male catheter being wrappable around a penis having said top lobe being positioned on top of the penis and having said bottom lobe being positioned on the bottom of the penis wherein said male catheter is configured to capture urine from the penis, said male catheter having a first surface and a second surface having said perimeter edge extending therebetween, said first lobe having an aperture extending through said first surface and said second surface wherein said aperture is configured to be aligned the external urethral orifice in the penis thereby facilitating urine to pass through said aperture, said aperture being positioned adjacent to an intersection between said first lobe and said peninsula, said male catheter being infused with aloe vera for reducing irritation caused by wearing said male catheter;
a first adhesive layer being bonded to said male catheter, said first adhesive layer adhesively engaging the penis when said male catheter is wrapped around the penis to retain said male catheter on the penis, said first adhesive layer being positioned on said first surface of said male catheter, said first adhesive layer completely covering said first surface;
a first tube being fluidly coupled to said male catheter wherein said first hose is configured to receive the urine from the penis, said first tube having a first end and a second end, said aperture in said male catheter insertably receiving said first end having said first tube being bonded to said male catheter;
a first collection bag being fluidly coupled to said first tube wherein said first collection bag is configured to capture the urine from the penis, said first collection bag having an inlet being integrated therein, said inlet having said second end of said tube being fluidly coupled thereto;
a female catheter being positionable within the labia minora of a female patient wherein said female catheter is configured to capture urine from the female patient, said female catheter having a primary surface, a secondary surface and an outer edge extending therebetween, said outer edge being continuously arcuate about a center point of said female catheter, said female catheter being longitudinally elongated such that said female catheter has an ovoid shape having a first end and a second end wherein said female catheter is configured to cover the vaginal opening and the urethra when said female catheter is worn, said female catheter having an aperture extending through said primary surface and said secondary surface wherein said aperture in said female catheter is configured to have urine pass therethrough, said aperture in said female catheter being aligned with a longitudinal axis of said female catheter, said aperture in said female catheter being positioned closer to said first end than said second end of said ovoid shape defined by said female catheter, said female catheter being infused with aloe vera for reducing irritation caused by wearing said female catheter;
a second adhesive layer being bonded to said female catheter, said second adhesive layer adhesively engaging the labia minora when said female catheter is positioned thereagainst to retain said female catheter on the labia minora, said second adhesive layer being positioned on said secondary surface of said female catheter, said second adhesive layer completely covering said secondary surface;
a second tube being fluidly coupled to said female catheter wherein said second hose is configured to receive the urine from the female patient, said second tube having a first end and a second end, said aperture in said female catheter insertably receiving said second end of said second tube having said second tube being bonded to said female catheter; and
a second collection bag being fluidly coupled to said second tube wherein said second collection bag is configured to capture the urine from the female patient, said second collection bag having an inlet being integrated therein, said inlet on said second collection bag having said second end of said second tube being fluidly coupled thereto.
